(19) United States
(12) Patent Application Publication
(10) Pub. No.: US 2019/0264251 A1
(43) Pub. Date: Aug. 29, 2019

(54) METHOD FOR MANUFACTURING THREE-DIMENSIONAL CELL CULTURE SUPPORT HAVING DOUBLE CROSSLINK, AND CASTING TRAY FOR MANUFACTURING THREE-DIMENSIONAL CELL CULTURE SUPPORT

(71) Applicants: Mi Sun CHA, Seoul (KR); MEDIFAB CO., LTD., Seoul (KR)

(72) Inventor: Mi Sun CHA, Seoul (KR)

(21) Appl. No.: 16/320,437

(22) PCT Filed: Jul. 19, 2017
(86) PCT No.: PCT/KR2017/007767

§ 371 Date: Jan. 24, 2019

(30) Foreign Application Priority Data
Jul. 25, 2016 (KR) 10-2016-0094327
Jun. 8, 2017 (KR) 20-2017-0002879

(51) Int. Cl. C12Q 1/04; C12N 5/00; C12N 5/09; C12N 5/095

(52) U.S. Cl. CPC C12Q 1/045; C12N 5/0068; C12N 5/0062; C12N 5/0694; C12N 5/0695; C12N 5/0018; C12N 2537/10; C12N 2533/54; C12N 2533/72; C12N 2533/76; C12N 2533/40; C12N 2533/74; C12N 2535/00; C12N 2513/00

(57) ABSTRACT

The present disclosure relates to a method for manufacturing a three-dimensional cell culture support having a double crosslink, and a casting tray for manufacturing the three-dimensional cell culture support, wherein the method for manufacturing the three-dimensional cell culture support having the double crosslink includes: producing a cell mixed hydrogel; manufacturing a casting gel mold in a three-dimensional shape; and manufacturing a structure gelated in a three-dimensional shape, and the casting tray for manufacturing the three-dimensional cell culture support includes: a tray part including a groove accommodating a gel solution; a mold part covering the tray part; and a mold protrusion provided on the mold part and inserted into the groove when the mold part covers the tray part.

### TECHNICAL FIELD

The present disclosure relates to a method for manufacturing a three-dimensional cell culture support having a double crosslink, and a casting tray for manufacturing the three-dimensional cell culture support, and more particularly, to a method for quickly and easily manufacturing a three-dimensional cell culture support having a double crosslink by gelating a hydrogel by a one-step method of applying ionic and physical crosslinks simultaneously, and a casting tray for manufacturing the three-dimensional cell culture support by manufacturing a casting gel mold in a three-dimensional shape through a mold part having a mold protrusion and manufacturing the three-dimensional cell culture support through the three-dimensional casting gel mold.

### BACKGROUND ART

During the past century, scientists cultured cells using flat two-dimensional Petri dishes and conducted studies on signaling, cytodifferentiation, etc. using cells cultured by the method in many laboratories.

When cells are cultured through a two-dimensional Petri dish, the cells change shape or are divided on the glass bottom of the Petri dish; however, cells in a real living body do not grow in this way.

When cells grow in a three-dimensional cell culture environment that is similar to the inside of a living body, greatly different gene expression may appear compared to when the cells are cultured two-dimensionally on the Petri dish. That is, since the same study may show different experimental results according to cell culture methods, applying experimental results using cells to the treatment of disease requires a lot of attention.

A hydrogel, which is a hydrophilic network structure material in which a polymer chain forms a three-dimensional structure, generally contains a large amount of water and has intermediate characteristics between liquid and solid. Therefore, the hydrogel can be used as a tissue regeneration support capable of replacing tissue or an organ when a body part is damaged or loses its function. The hydrogel has inherent properties of absorbing a large amount of water in an aqueous solution or under an aqueous environment to swell due to the hydrophile property of the ingredients, but not dissolving due to the crosslink structure. Accordingly, it is possible to manufacture hydrogels with various shapes and properties according to ingredients and manufacturing methods.

Alginate and gelatin are ones of polymers capable of producing a hydrogel. Alginate is a hydrogel biomaterial widely used for a long time in tissue engineering, etc. Alginate is a natural polymer extracted from seaweed, and includes two kinds of uronic acid of B-D-mannuronic acid and a-L-glucuronic acid. When alginate is dissolved in distilled water, the alginate becomes liquid having viscosity, and when a polyvalent metal, such as Ca²⁺, Sr²⁺, or Ba²⁺, is added to alginate, particles are formed through gelation and crosslinking by ionic bonding between the metal ions and Na⁺ ions of glucuronic acid.

Alginate beads manufactured by the method are catching on as new cell culture technology for exactly reproducing responses of real living bodies through a cell culture method of mimicking the structure of a three-dimensional body organ, which is beyond the limits of existing two-dimensional cell culture technology. However, since alginate itself is inert biologically, it cannot be used for purposes, such as getting animal cells to grow or move thereon.

Gelatin, which is another polymer capable of forming a hydrogel, is a derived protein obtained by partial hydrolysis of collagen, which is a main protein ingredient of connective tissues, such as the bones, cartilage, skins, etc. of animals. Since gelatin has high biocompatibility and a nontoxic, biodegradable property, it is widely used in various industries, such as food, medicine, photography, and cosmetics.

Gelatin provides viscosity even at relatively low temperature and concentration, and a gelatin solution forms a clear, elastic, and thermoreversible gel when it cools. However, since gelatin is easily dissolved in an aqueous solution, crosslinking with a chemical material, such as formaldehyde or glutaraldehyde, is used to increase stability in an aqueous solution.

However, when even the slightest amount of such crosslinking agent ingredients remains, cytotoxicity appears, and when the ingredients are implanted into a living body, they may show toxicity on the surrounding organs. Therefore, studies for minimizing use of the crosslinking agent or for increasing the mechanical property of a support without using the crosslinking agent are urgently needed. Also, it is urgent to develop a cell culture support capable of implementing various three-dimensional shapes.

The present invention was invented by performing a national research and development project (Project No.: S2596884, Name of Dept.: Department of Small and Medium Venture Business, Research Management Agency: Small and Medium Business Technical Information Agency, Research Business Name: First step of industry-academia cooperation technology development business, Research Project Name: Establishment of base technology for development of 3D osteoporosis model alternative to animal experiment, Main Agency: Medifab Co., Ltd.) The present invention was supported by the Technology development Program (S2607243) funded by the Ministry of SMEs and Startups (MSS, Korea)

### DESCRIPTION OF EMBODIMENTS

### Technical Problem

Provided are a method for quickly and easily manufacturing a three-dimensional cell culture support having a double crosslink by gelating a hydrogel by a one-step method of applying ionic and physical crosslinks simultaneously, and a casting tray for manufacturing the three-dimensional cell culture support by manufacturing a casting gel mold in a three-dimensional shape through a mold part having a mold protrusion and manufacturing the three-dimensional cell culture support through the three-dimensional casting gel mold.

### Solution to Problem

According to an aspect of the present disclosure, there is provided a method for manufacturing a three-dimensional cell culture support having a double crosslink, including: producing a cell mixed hydrogel; manufacturing a casting gel mold in a three-dimensional shape; and dispersing the cell mixed hydrogel into the casting gel mold manufactured in the three-dimensional shape, and gelating the cell mixed hydrogel to manufacture a structure gelated in a three-dimensional shape.

The producing of the cell mixed hydrogel may include: mixing gelatin with alginate to prepare a mixed solution; filtering the mixed solution to produce a hydrogel; and mixing the hydrogel with cells.

The mixed solution may be one selected from a group consisting of apatite, cellulose, gellan, agarose, chitosan, keratin, and collagen, or a combination of two or more of apatite, cellulose, gellan, agarose, chitosan, keratin, and collagen, and the mixed solution may be one selected from a group consisting of a transforming growth factor (TGF), a vascular endothelial growth factor (VEGF), a fibroblast growth factor (FGF), an epidermal growth factor (EGF), a platelet-derived epidermal growth factor (PDGF), a hepatocyte growth factor (HGF), an insulin like growth factor (IGF), cytokine, and chemokine, or a combination of two or more of a transforming growth factor (TGF), a vascular endothelial growth factor (VEGF), a fibroblast growth factor (FGF), an epidermal growth factor (EGF), a platelet-derived epidermal growth factor (PDGF), a hepatocyte growth factor (HGF), an insulin like growth factor (IGF), cytokine, and chemokine.

The cells may be ones selected from a group consisting of cancer cells, stem cells, sensory cells, brain cells, reproductive cells, epithelial cells, immune cells, and bone cells, or a combination of two or more of cancer cells, stem cells, sensory cells, brain cells, reproductive cells, epithelial cells, immune cells, and bone cells, and the cancer cells may be one selected from a group consisting of a lung cancer cell line (BEAS2B cell), a stomach cancer cell line (AGS cell), and a cervical cancer cell line (HeLa cell).

The manufacturing of the casting gel mold in the three-dimensional shape may include: dissolving a biodegradable polymer in a divalent cation aqueous solution and adding a pH indicator changing the color according to a change of pH to produce a casting gel solution; and putting the casting gel solution in a tray part having a groove, inserting a mold part having a mold protrusion of a three-dimensional shape into the tray part, solidifying the casting gel solution, and removing the mold part to manufacture a three-dimensional casting gel mold.

The biodegradable polymer may be one selected from a group consisting of agarose, dextran, silica gel, and polyethylene glycol (PEG), or a combination of two or more of agarose, dextran, silica gel, and polyethylene glycol (PEG), and the divalent cation aqueous solution may be one selected from a group consisting of calcium chloride (CaCl₂), calcium sulfate (CaSO₄), and calcium carbonate (CaCO₃), or a mixed solution of two or more of calcium chloride (CaCl₂), calcium sulfate (CaSO₄), and calcium carbonate (CaCO₃). The pH indicator may be one selected from a group consisting of phenol red, bromthymol blue and phenolphthalein, and the pH indicator may change the color under an acidic or alkaline condition.

The manufacturing of the structure gelated in the three-dimensional shape may include dispensing the cell mixed hydrogel into the three-dimensional casting gel mold to gelate the cell mixed hydrogel at −4° C. to 37° C. for 15 minutes to 25 minutes.

According to another aspect of the present disclosure, there is provided a casting tray for manufacturing a three-dimensional cell culture support, including: a tray part formed in a plate shape and including a groove extending in a longitudinal direction of the plate shape in the inside of the plate shape, wherein a gel solution is accommodated in the groove; and a mold part formed in a plate shape and covering the tray part after the gel solution is accommodated in the groove of the tray part, wherein the mold part may include a mold protrusion protruding toward the groove from the mold part, and inserted into the groove when the mold part covers the tray part.

The mold protrusion may be formed by combining at least one shape among a polyhedron, a cone, a cylinder, a hemisphere, and a sphere shape, the mold protrusion may include an embossed part embossed in a surface of the mold protrusion or an engraved part engraved in the surface of the mold protrusion, and the embossed part and the engraved part may be formed in a spiral shape.

In the groove of the tray part, a tray protrusion protruding from the groove may be formed, and in the groove of the tray part, a tray groove engraved in a predetermined shape from the groove may be formed.

The mold part may be provided as a plurality of mold parts having different shapes of mold protrusions, and the plurality of mold parts may cover the tray part sequentially.

### Advantageous Effects of Disclosure

By applying a method of manufacturing a three-dimensional cell culture support having a double crosslink, according to the present disclosure, to gelate a hydrogel by a one-step method of applying ionic and physical crosslinks simultaneously, a three-dimensional cell culture support can be quickly and easily formed in a uniform shape, progression and completion of gelation can be checked, a customized three-dimensional culture platform can be manufactured by applying various three-dimensional shapes of casing molds, and also, excellent bioaffinity can be obtained since no chemical crosslinking agent is used.

Through the mold part of the casting tray having the mold protrusion according to the present disclosure, various shapes of three-dimensional cell culture supports can be formed, and when a plurality of mold parts having different shapes or sizes of mold protrusions are used, a cell mixed hydrogel having a layered structure can be formed to mimic a tissue shape more similar to a living body.

### BRIEF DESCRIPTION OF DRAWINGS

FIG. 1 shows a process of manufacturing a casting gel mold according to an embodiment of the present disclosure.

FIG. 2 shows gel casting processes according to Embodiments 1 to 3.

FIG. 3 shows gel casting processes according to the Embodiments 1 to 3.

FIG. 4 shows biocompatibility evaluation of a three-dimensional cell culture support having a double crosslink, manufactured according to Embodiment 1.

FIG. 5 shows biocompatibility evaluation of a three-dimensional cell culture support having a double crosslink, manufactured according to Embodiment 2.

FIG. 6 shows biocompatibility evaluation of a three-dimensional cell culture support having a double crosslink, manufactured according to Embodiment 3.

FIG. 7 is a perspective view of a casting tray according to an embodiment of the present disclosure.

FIG. 8 shows a tray part according to an embodiment of the present disclosure.

FIG. 9 shows a mold part according to an embodiment of the present disclosure.

FIG. 10 shows mold protrusions according to an embodiment of the present disclosure, in which embossed parts and engraved parts are formed.

FIG. 11 shows a state in which a casting gel solution is accommodated in a tray part and the tray part is covered by a mold part, according to an embodiment of the present disclosure.

FIGS. 12 and 13 show a process for manufacturing a three-dimensional cell culture support having a layered structure through a plurality of mold parts, according to an embodiment of the present disclosure.

FIG. 14 shows a three-dimensional cell culture support having a layered structure, manufactured through a plurality of mold parts, according to an embodiment of the present disclosure.

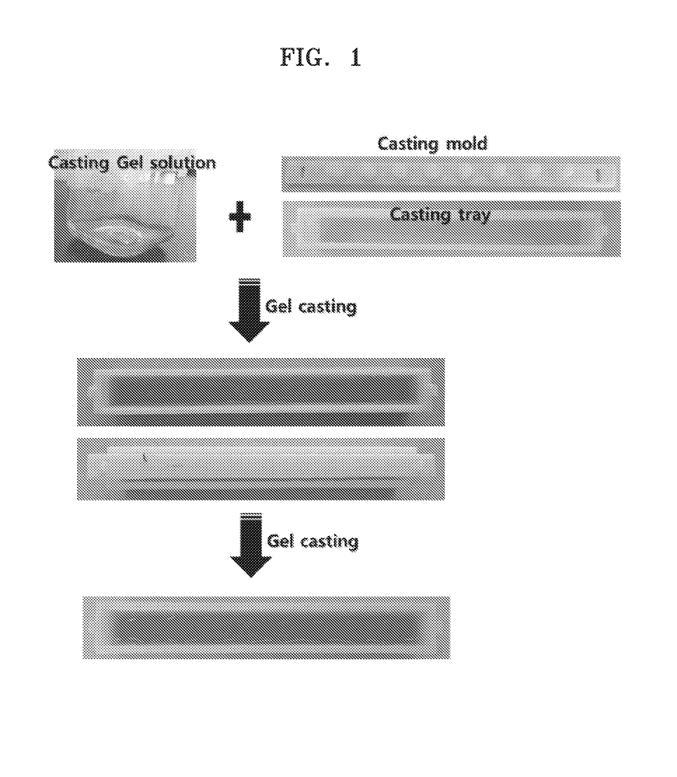

### MODE OF DISCLOSURE

Hereinafter, a method for manufacturing a three-dimensional cell culture support having a double crosslink, according to the present disclosure, and a casting tray for manufacturing the three-dimensional cell culture support will be described in more detail.

Inventors of the method for manufacturing the three-dimensional cell culture support having the double crosslink, according to the present disclosure, found that a three-dimensional cell culture support having a double crosslink can be quickly and easily formed by gelating a hydrogel by a one-step method of applying ionic and physical crosslinks simultaneously, thereby completing the present disclosure.

The method for manufacturing the three-dimensional cell culture support having the double crosslink, according to the present disclosure, may include operations of: producing a cell mixed hydrogel; manufacturing a three-dimensional casting gel mold; and dispensing the cell mixed hydrogel into the three-dimensional casting gel mold to manufacture a three-dimensional structure gelated with a double crosslink.

Operation of producing the cell mixed hydrogel may include operations of: mixing gelatin with alginate to prepare a mixed solution; filtering the mixed solution to produce a hydrogel; and mixing the hydrogel with cells, although not limited thereto.

The mixed solution may be any one selected from a group consisting of apatite, cellulose, gellan, agarose, chitosan, keratin, and collagen, or a combination of two or more of the above-mentioned materials, although not limited thereto.

Particularly, when the mixed solution further includes apatite, the effects of enhancing physical properties and increasing cytotroprism on the osteogenic cell line may be expected.

Also, the mixed solution may be any one selected from a group consisting of a transforming growth factor (TGF), a vascular endothelial growth factor (VEGF), a fibroblast growth factor (FGF), an epidermal growth factor (EGF), a platelet-derived epidermal growth factor (PDGF), a hepatocyte growth factor (HGF), an insulin like growth factor (IGF), cytokine, and chemokine, or a combination of two or more of the above-mentioned factors, although not limited thereto.

The cells may be any ones selected from a group consisting of cancer cells, stem cells, sensory cells, brain cells, reproductive cells, epithelial cells, immune cells, and bone cells, or a combination of two or more of the above-mentioned cells, although not limited thereto.

The cancer cells may be any one selected from a group consisting of a lung cancer cell line (BEAS2B cell), a stomach cancer cell line (AGS cell), and a cervical cancer cell line (HeLa cell), or a combination of two or more of the above-mentioned cell lines, although not limited thereto.

Operation of manufacturing the three-dimensional casting gel mold may include operations of: dissolving a biodegradable polymer in an alginic acid aqueous solution, a divalent cation aqueous solution, or a mixture thereof and then adding a pH indicator changing the color according to a change of pH to produce a casting gel solution; and putting the casting gel solution in a tray part 110 having a groove, inserting a mold part 120 having a mold protrusion 121 of a three-dimensional shape into the tray part 110, solidifying the casting gel solution, and then removing the mold part 120 to manufacture a three-dimensional casting gel mold, although not limited thereto.

A casting tray for manufacturing the three-dimensional cell culture support according to the present disclosure may include the tray part 110 and the mold part 120, and may be used in the operation of manufacturing the three-dimensional casting gel mold. A detailed description about the casting tray will be described later.

The biodegradable polymer may be any one selected from a group consisting of agarose, dextran, silica gel, and polyethylene glycol (PEG), or a combination of two or more of the above-mentioned materials, although not limited thereto.

The divalent cation aqueous solution may be any one selected from a group consisting of calcium chloride (CaCl₂), calcium sulfate (CaSO₄), and calcium carbonate (CaCO₃), or a combination of two or more of the above-mentioned materials, although not limited thereto.

The pH indicator may be any one selected from a group consisting of phenol red, bromthymol blue, and phenolphthalein, although not limited thereto.

The pH indicator may change the color under an acidic or alkaline condition, although not limited thereto.

The operation of manufacturing the structure gelated in the three-dimensional shape may be the operation of dispensing the cell mixed hydrogel into the three-dimensional casting gel mold to gelate the cell mixed hydrogel at −4° C. to 25° C. for 15 minutes to 25 minutes, although not limited thereto.

Under the gelation condition, when the temperature is lower than −4° C. or higher than 37° C., the viability of the cells deteriorates rapidly. Also, when the temperature exceeds 25° C., the physical crosslink of gelatin is interfered. Therefore, the gelation may be performed preferably at −4° C. to 25° C. or at −4° C. to 37° C. Particularly, considering that gelation of a structure including cells is derived, the gelation may be performed more preferably at 4° C.

The three-dimensional casting gel mold may be characterized in that gelation according to a one-step method of simultaneously performing a double crosslink including an ionic crosslink using diffusion and a physical crosslink using thermo-sensitivity and gelation to which various and complicated three-dimensional shapes are applied are possible and that progression and completion of gelation can be checked through a change in color of the casting gel mold.

Also, the present disclosure may provide a three-dimensional cell culture support having a double crosslink, manufactured by the above-described manufacturing method.

Hereinafter, the present disclosure will be described in more detail by the following embodiments. However, the present disclosure is not limited to the embodiments.

### Embodiment 1

### Manufacturing of a Three-Dimensional Cell Culture Support Having a Double Crosslink

### 1. Preparing Materials

Agarose (Sigma Aldrich, U.S.A), CaCl₂ (Sigma Aldrich, U.S.A), a phenol red solution (Sigma Aldrich, U.S.A), and a Dulbecco's phosphate buffered saline (hereinafter, simply referred to as “DPBS”, Wellgin, Korea) solution were prepared.

Gelatin (type A from porcine skin) and alginate powder were purchased from Sigma Aldrich (U.S.A).

### 2. Producing a Cell Mixed Hydrogel

3 g Gelatin was added in a 100 ml DPBS solution, and then dissolved completely using a hot plate stirrer (DAIHAN SCIENTIFIC CO., Ltd., Korea) at 60° C. to produce a gelatin solution. Thereafter, 2 g of alginate powder was added in the gelatin solution, and then dissolved completely using the hot plate stirrer for one hour to produce an alginate-gelatin mixed solution.

The alginate-gelatin mixed solution was filtered using a syringe filter (Macherey-Nagel, Germany) of 0.45 μm to produce a hydrogel, and the hydrogel was stored in a sterilized bottle to be used.

The hydrogel that is to be gelated in a three-dimensional shape was pre-warmed up at 37° C., and then, a lung cancer cell line (hereinafter, referred to as a “BEARS2B cell”, Korean Cell Line Bank) of 2×10⁶ cell/ml was carefully mixed with the pre-warmed hydrogel to produce a cell mixed hydrogel.

### 3. Manufacturing a Casting Gel Mold

0.5% Agarose was dissolved in a 300 mM CaCl₂ solution, and then, a phenol red solution of 5 mg/L was added to produce a casting gel solution.

The casting gel solution was put into a casting tray, then a casting mold formed in a three-dimensional hemispherical shape was inserted into the casting tray, the casting gel solution was completely solidified, and then the casting mold was carefully removed to manufacture a casting gel mold in a three-dimensional hemispherical shape.

### 4. Gel Casting

The cell mixed hydrogel of 40 ml was dispersed in the casting gel mold, gelation was performed at 4° C. (or a state in which it is put in an ice bucket) until the color of the phenol red solution changes from red (alkalinity) to yellow (acidity), and the resultant structure gelated in the three-dimensional hemispherical shape was taken out of the mold using a forcep to manufacture a three-dimensional cell culture support having a double crosslink.

### Embodiment 2

### Manufacturing a Three-Dimensional Cell Culture Support Having a Double Crosslink

A three-dimensional cell culture support having a double crosslink was manufactured under the same conditions as Embodiment 1 except that the cervical cancer cell line (hereinafter, referred to as “HeLa cell”, Korean Cell Line Bank), instead of the lung cancer cell line, is used.

### Embodiment 3

### Manufacturing a Three-Dimensional Cell Culture Support Having a Double Crosslink

A three-dimensional cell culture support having a double crosslink was manufactured under the same conditions as Embodiment 1 except that tricalcium phosphate (hereinafter, referred to as “TCP”, Ca₃(PO₄)₂, Sigma Aldrich, U.S.A.) of 2 weight % was further added in the alginate-gelatin mixed solution of Embodiment 1 to use the osteoblastic cell line (hereinafter, referred to as “MC3T3-E1”, Korean Cell Line Bank).

### Experimental Example 1

### Evaluation of Gelation According to a Change of pH

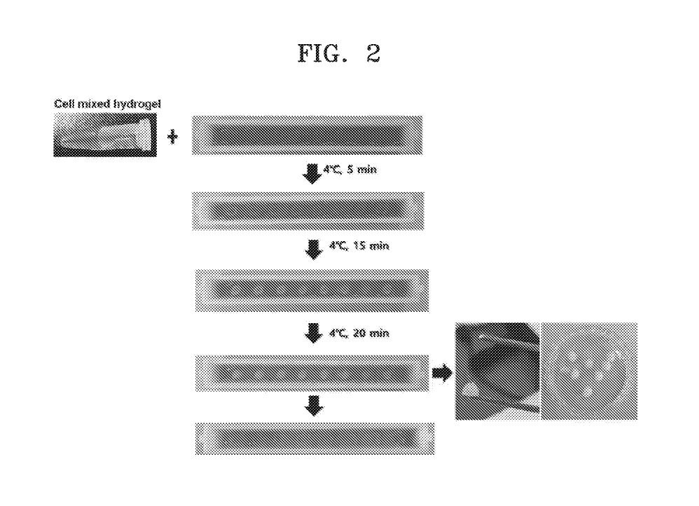

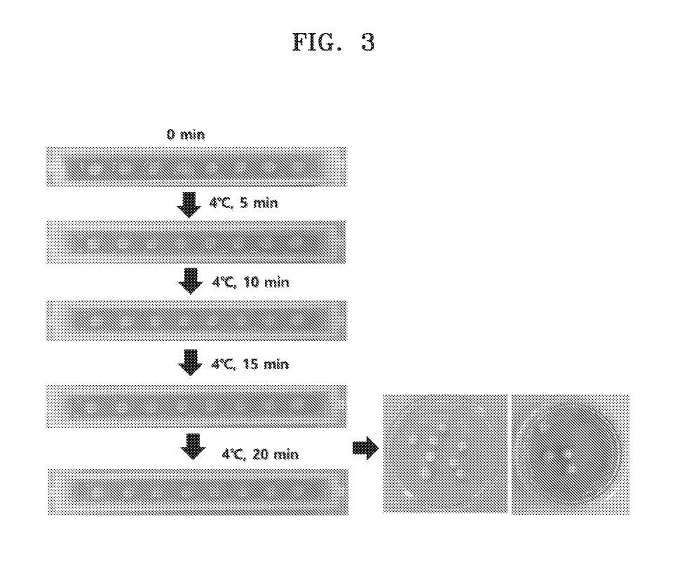

Referring to FIGS. 2 and 3, in the gel casting operation according to Embodiments 1 to 3, Ca²⁺ was diffused in the mold to gelate alginate so that the pH of the mold changed to acidity, and accordingly, the color of the mold was changed from red (alkalinity) to yellow (acidity) by the phenol red solution, which is a pH indicator. Through the change in color of the mold, it could be easily confirmed that gelation occurred.

### Experimental Example 2

### Evaluation on Biocompatibility

Biocompatibility of the three-dimensional cell culture support having the double crosslink, manufactured according to Embodiments 1 to 3, was evaluated.

A Dulbecco's Modified Eagle's Medium (DMEM)/low glucose medium including a fetal bovine serum (hereinafter, referred to as “FBS”, Gibco, U.S.A.) of 10% was added in the three-dimensional cell culture support having the double crosslink manufactured by Embodiments 1 to 3, and cultured at 37° C.

A 100 mM sodium citrate solution of 200 mg was added in the cultured three-dimensional support for each culture time, the support was destructurized at 37° C. for 30 minutes, then 20 ml of EZ-cytox (DAEILLAB SERVICE, Korea) was put to react at 37° C. for one hour, and thereafter, absorbance was measured at 450 nm (Molecular Device, U.S.A.).

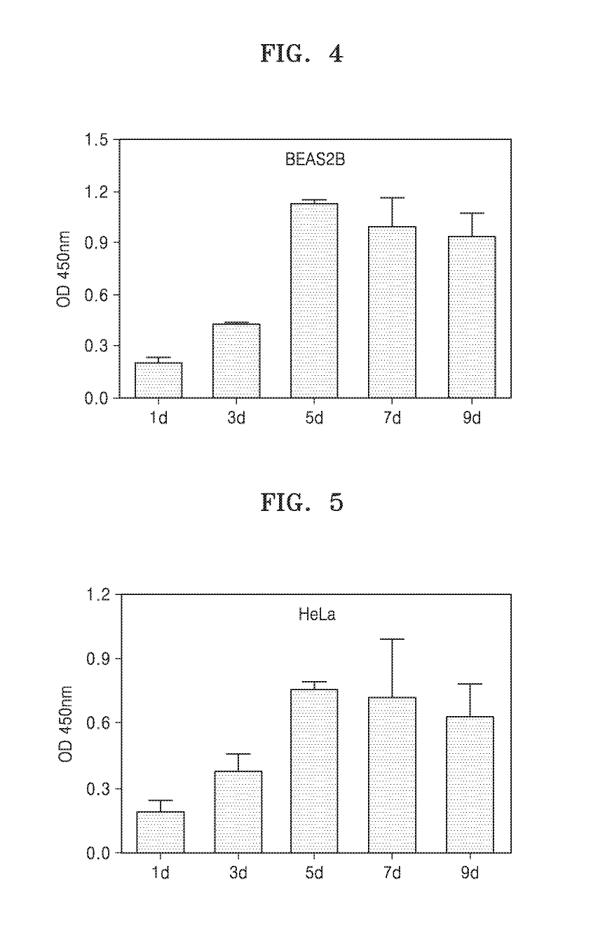

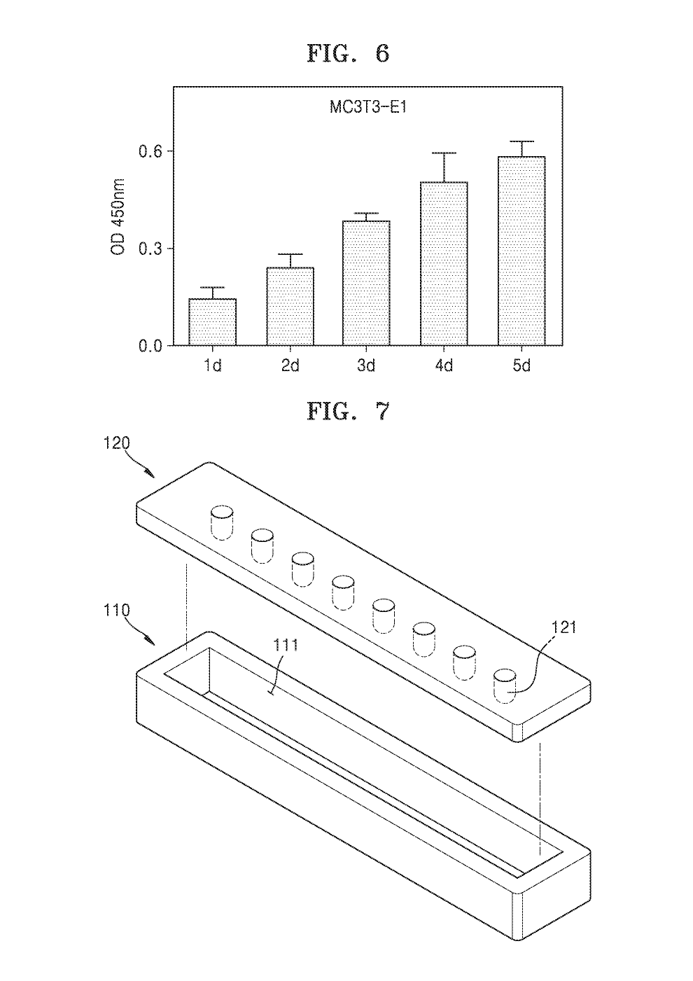

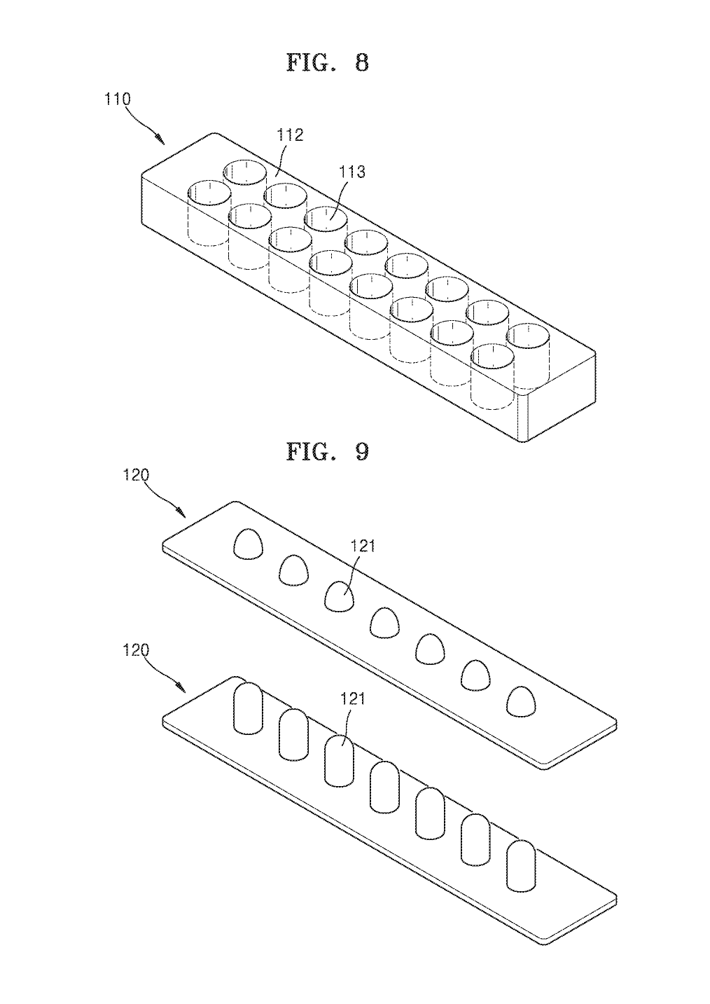

Referring to FIGS. 4 to 6, it could be confirmed that all cell lines of Embodiments 1 to 3 were multiplied normally, and most multiplied in fifth to seventh days. Therefore, the biocompatibility of the three-dimensional cell culture support having the double crosslink, manufactured according to Embodiments 1 to 3, was verified.

In the operation of manufacturing the three-dimensional casting gel mold, a casting tray for manufacturing a three-dimensional cell culture support may be used, and the casting tray for manufacturing the three-dimensional cell culture support will be described in detail with reference to the accompanying drawings.

Referring to FIG. 7, the casting tray for manufacturing the three-dimensional cell culture support may include a tray part 110, a mold part 120, and a mold protrusion 121.

The tray part 110 may be formed in a plate shape, and a groove 111 may be formed in the inside of the plate shape in such a way to extend in a longitudinal direction of the plate shape. More specifically, the groove 111 of the tray part 110 may accommodate a gel solution. The gel solution may be the casting gel solution described above. The casting gel solution has been described above, and accordingly, a further description thereof will be omitted.

However, a gel solution that is accommodated in the groove 111 is not limited to the casting gel solution, and various kinds of gel solutions may be accommodated in the groove 111. For example, the casting gel solution may be accommodated in the groove 111 and gelated, and thereafter, a cell mixed hydrogel may be accommodated on the casting gel solution (see FIG. 13).

The tray part 110 may be formed in various shapes as long as the groove 111 capable of accommodating a gel solution can be formed in the tray part 110. For example, the tray part 110 may be formed in the shape of a rectangular plate, and the groove 111 may extend along the longer side (in the longitudinal direction of the plate) of the tray part 110 formed in the rectangular shape.

The groove 111 of the tray part 110 may be formed in various shapes. In the groove 111 of the tray part 110, a tray protrusion 112 may protrude from the groove 111, and a tray groove 113 may be engraved in a predetermined shape from the groove 111. That is, the groove 111 may have a shape defined by the tray protrusion 112 and the tray groove 113 protruding and engraved from the groove 111.

When the groove 111 has a shape, a gel solution accommodated in the groove 111 may be gelated in a shape corresponding to the groove 111. For example, when the casting gel solution is accommodated in the groove 111 having a shape, the casting gel solution may be gelated in a three-dimensional shape corresponding to the groove 111, and a casting gel mold having a three-dimensional shape may be formed.

More specifically, the groove 111 may be a groove extending in a rectangular shape, as shown in FIG. 7, and also, as shown in FIG, 8, the groove 111 may be in the shape of a well plate, wherein the well plate may be formed by combining the tray protrusion 112 with the tray groove 113 in the groove 111. The shape of the groove 111 is not limited to this, and the groove 111 may be formed in various shapes as long as it can accommodate a gel solution.

The mold part 120 may be formed in a plate shape, and cover the tray part 110 after a gel solution is accommodated in the groove 111 of the tray part 110. That is, the mold part 120 may be a cover for covering the tray part 110. Therefore, the mold part 120 may be shaped to cover the tray part 110 in correspondence to the shape of the tray part 110.

The mold part 120 may protrude toward the groove 111, and include the mold protrusion 121 that is inserted into the groove 111 when the mold part 120 covers the tray part 110. The mold protrusion 121 may be positioned at a location at which it is inserted into the groove 111 when the tray part 110 is covered by the mold part 120 after a gel solution is accommodated in the groove 111.

When the mold protrusion 121 is inserted into the groove 111, the mold protrusion 121 may provide a predetermined shape to the gel solution accommodated in the groove 111. The gel solution may be a material that can change the shape before it is gelated, Therefore, the gel solution may be changed to a shape corresponding to that of the mold protrusion 121 by the mold protrusion 121, and may be gelated and solidified in the changed shape.

That is, when the casting gel solution as the gel solution is accommodated in the groove 111, the casting gel solution may be gelated in a shape corresponding to that of the mold protrusion 121 to form a three-dimensional casting gel mold 130.

To change a three-dimensional shape that is formed in the casting gel mold 130, the mold protrusion 121 may be formed in various shapes. Referring to FIG. 9, the mold protrusion 121 may be formed by combining at least one shape among a polyhedron, a cone, a cylinder, a hemisphere, and a sphere shape. More specifically, the mold protrusion 121 may be in the shape of a hemisphere, and the mold protrusion 121 may be in the shape of a cylinder whose top is in the shape of a hemisphere.

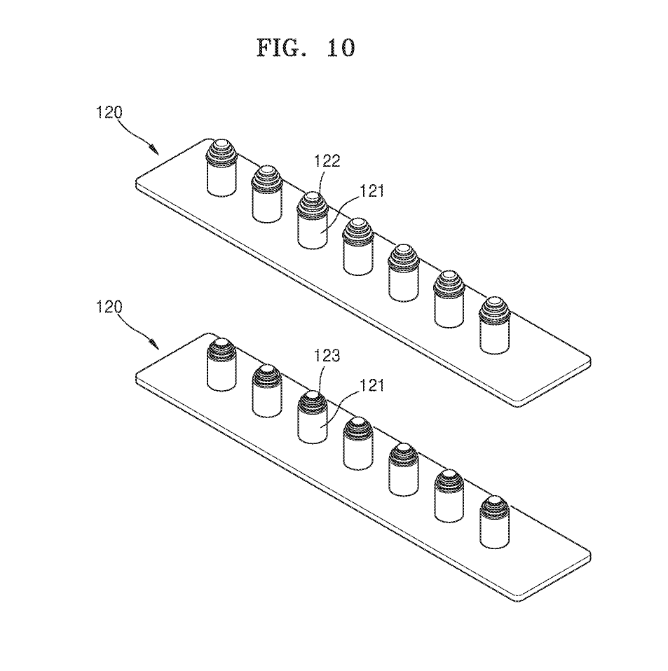

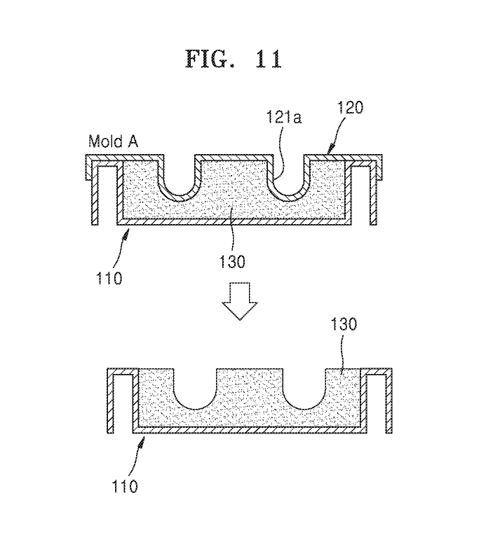

Also, referring to FIG. 10, the mold protrusion 121 may include an embossed part 122 embossed in the surface or an engraved part 123 engraved in the surface. The embossed part 122 and the engraved part 123 may be formed in a spiral shape, and by forming the embossed part 122 and the engraved part 123, a spiral shape of an embossed part and an engraved part may also be formed in a three-dimensional shape formed in the casting gel mold. More specifically, when the embossed part 122 is formed in the surface of the mold protrusion 121, an engraved part may be formed in the casting gel mold, and when the engraved part 123 is formed in the surface of the mold protrusion 121, an embossed part may be formed in the casting gel mold.

The embossed part 122 and the engraved part 123 may be formed in the top of the mold protrusion 121 or in the entire mold protrusion 121. That is, the mold protrusion 121 may be formed by combining various shapes (for example, a polyhedron, a cone, a cylinder, a hemisphere, a sphere, an engraved part, and an embossed part) according to a desired three-dimensional shape of the casting gel mold 130.

Also, a plurality of mold protrusions 121 may be formed in the mold part 120. The plurality of mold protrusions 121 may have the same shape or different shapes as necessary.

Before the casting gel solution accommodated in the groove 111 is gelated, the shape of the casing gel solution may change. Referring to FIG. 11, after the casting gel solution is accommodated in the groove 111, the tray part 110 may be covered by the mold part 120. By covering the tray part 110 with the mold part 120, the mold protrusion 121 of the mold part 120 may be inserted into the groove 111, and the casting gel solution may change to a shape corresponding to the mold protrusion 121.

When the casting gel solution is gelated in a state in which the mold protrusion 121 is inserted in the groove 111, the casting gel solution may form the casting gel mold 130 in the shape corresponding to the mold protrusion 121.

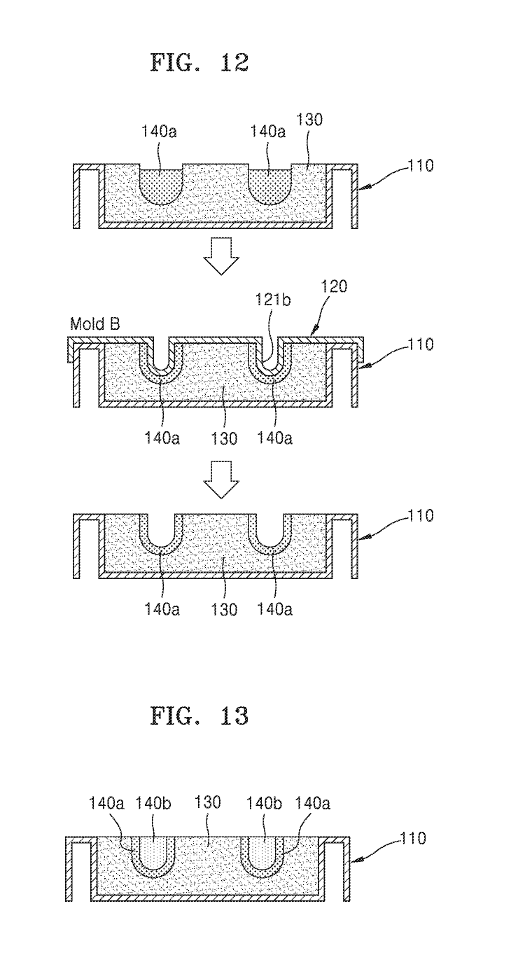

Referring to FIG. 12, a cell mixed hydrogel 140a may be dispersed in the casting gel mold 120, and gelated at −4° C. to 25° C. for 15 minutes to 25 minutes. After the cell mixed hydrogel 140a is gelated, the cell mixed hydrogel 140a may be separated from the casting gel mold 130 to manufacture a three-dimensional cell culture support.

Herein, the cell mixed hydrogels 140a and 140b are the same as the cell mixed hydrogel used in the above-described method for manufacturing the three-dimensional cell culture support having the double crosslink. The cell mixed hydrogel has been described above, and accordingly, a detailed description thereof will be omitted. Also, the cell mixed hydrogel may be one of various kinds of hydrogels, and the hydrogel 140a may be a different kind of hydrogel from the hydrogel 140b.

The mold part 120 according to the present disclosure may be provided as a plurality of mold parts 120 having different shapes, so that the plurality of mold parts 120 may cover the tray part 110 sequentially to form cell mixed hydrogels 140a and 140b having a layered structure.

More specifically, the mold part 120 may be provided as two or more mold parts 120 having different shapes. A process of forming the cell mixed hydrogels 140a and 140b having a layered structure through the plurality of mold parts 120 will be described as follows.

Referring to FIG. 11, after the casting gel solution is accommodated in the groove 111, the tray part 110 may be covered by the mold part 120. The mold part 120 may include a mold protrusion A 121a. By covering the tray part 110 with the mold part 120, the casting gel solution may change to a shape corresponding to the mold protrusion A 121a to form the casting gel mold 130.

Thereafter, referring to FIG. 12, a hydrogel 140a in which first cells are mixed may be dispersed into the casting gel mold 120. Thereafter, a mold part 120 including a mold protrusion B 121b having a different shape from the mold protrusion A 121a used to manufacture the casting gel mold 130 may be prepared, wherein the mold protrusion B 121b may be smaller than the mold protrusion A 121a and be inserted into a location at which the hydrogel 140a in which the first cells are mixed is positioned.

The hydrogel 140a in which the first cells are mixed may be dispersed into the casting gel mold 130, and the mold protrusion B 121b may be inserted into the groove 111 before the hydrogel 140a in which the first cells are mixed is gelated. Since the hydrogel 140a in which the first cells are mixed has not yet been gelated, the hydrogel 140a in which the first cells are mixed may be gelated in a shape corresponding to the mold protrusion B 121b.

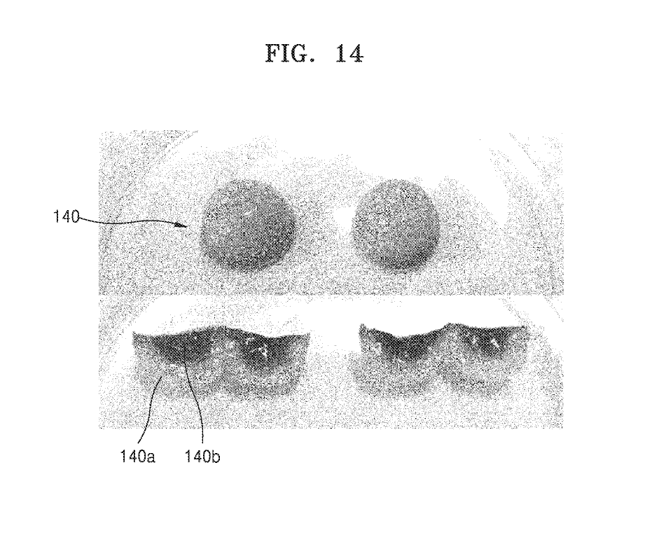

Referring to FIG. 13, after the hydrogel 140a in which the first cells are mixed is completely gelated, a hydrogel 140b in which second cells are mixed may be dispersed on the hydrogel 140a gelated in the shape corresponding to the mold protrusion B 121b, and then, the hydrogel 140b may be gelated. In this way, the hydrogels 140a and 140b in which the cells are mixed may form a layered structure. FIG. 14 shows the cell mixed hydrogels 140a and 140b having a layered structure, separated from the casting gel mold 130.

However, the layered structure of the cell mixed hydrogels 140a and 140b is not limited to a two-layered structure, and by using a plurality of mold parts 120 formed in various shapes, cell mixed hydrogels may be formed in a three or more layered structure.

The casting tray used to manufacture the three-dimensional cell culture support according to the present disclosure as described above may provide the following effects.

The casting tray according to the present disclosure may form a three-dimensional cell culture support in various shapes. By changing the shape of the mold protrusion 121, the shape of the casting gel mold 130 may change, thereby determining the shape of the hydrogel 140 in which the cells are mixed, the hydrogel 140 dispersed in the casting gel mold 120 and gelated.

Also, by using the plurality of mold parts 120 having different shapes, the hydrogels 140a and 140b in which the cells are mixed, the hydrogels 140a and 140b having a layered structure, may be formed. The hydrogels 140a and 140b in which the cells are mixed and which have a layered structure may include two or more kinds of cells. In this way, various three-dimensional cell culture supports may be manufactured, and also, a three-dimensional cell culture support in which various cells are mixed may be manufactured.

While the present disclosure has been described with reference to exemplary embodiments shown in the drawings, it will be understood by one of ordinary skill in the art that various modifications and equivalent embodiments may be made from the embodiments. Thus, the scope of the present disclosure should be defined by the appended claims.

1. A method for manufacturing a three-dimensional cell culture support having a double crosslink, comprising:
producing a cell mixed hydrogel;
manufacturing a casting gel mold in a three-dimensional shape; and
dispersing the cell mixed hydrogel into the casting gel mold manufactured in the three-dimensional shape and gelating the cell mixed hydrogel to manufacture a structure gelated in a three-dimensional shape.

2. The method of claim 1, wherein the producing of the cell mixed hydrogel comprises:
mixing gelatin with alginate to prepare a mixed solution;
filtering the mixed solution to produce a hydrogel; and
mixing the hydrogel with cells.

3. The method of claim 2, wherein the mixed solution is one selected from a group consisting of apatite, cellulose, gellan, agarose, chitosan, keratin, and collagen, or, a combination of two or more of apatite, cellulose, gellan, agarose, chitosan, keratin, and collagen.

4. The method of claim 2, wherein the mixed solution is one selected front a group consisting of a transforming growth factor (TGF), a vascular endothelial growth factor (VEGF), a fibroblast growth factor (FGF), an epidermal growth factor (EGF), a platelet-derived epidermal growth factor (PDGF), a hepatocyte growth factor (HGF), an insulin like growth factor (IGF), cytokine, and chemokine, or a combination of two or more of a transforming growth factor (TGF), a vascular endothelial growth factor (VEGF), a fibroblast growth factor (FGF), an epidermal growth factor (EGF), a platelet-derived epidermal growth factor (PDGF), a hepatocyte growth factor (HGF), an insulin like growth factor (IGF), cytokine, and chemokine.

5. The method of claim 2, wherein the cells are ones selected from a group consisting of cancer cells, stem cells, sensory cells, brain cells, reproductive cells, epithelial cells, immune cells, and bone cells, or a combination of two or more of cancer cells, stem cells, sensory cells, brain cells, reproductive cells, epithelial cells, immune cells, and bone cells.

6. The method of claim 5, wherein the cancer cells are ones selected from a group consisting of a lung cancer cell line (BEAS2B cell), a stomach cancer cell line (AGS cell), and a cervical cancer cell line (HeLa cell).

7. The method of claim 1, wherein the manufacturing of the casting gel mold in the three-dimensional shape comprises:
dissolving a biodegradable polymer in a divalent cation. aqueous solution and adding a pH indicator changing the color according to a change of pH to produce a casting gel solution; and
putting the casting gel solution in a tray part having a groove, inserting a mold part having a mold protrusion of a three-dimensional shape into the tray part, solidifying the casting gel solution, and removing the mold part to manufacture a three-dimensional casting gel mold.

8. The method of claim 7, wherein the biodegradable polymer is one selected from a group consisting of agarose, dextran, silica gel, and polyethylene glycol (PEG), or a combination of two or more of agarose, dextran, silica gel, and polyethylene glycol (PEG).

9. The method of claim 7, wherein the divalent cation aqueous solution is one selected from a group consisting of calcium chloride (CaCl₂), calcium sulfate (CaSO₄), and calcium carbonate (CaCO₃), or a mixed solution of two or more of calcium chloride (CaCl₂), calcium sulfate (CaSO₄), and calcium carbonate (CaCO₃).

10. The method of claim 7, wherein the pH indicator is one selected front a group consisting of phenol red, bromthymol blue, and phenolphthalein.

11. The method of claim 7, wherein the pH indicator changes the color under an acidic or alkaline condition.

12. The method of claim 1, wherein the manufacturing of the structure gelated in the three-dimensional shape comprises dispensing the cell mixed hydrogel into the three-dimensional casting gel mold to gelate the cell mixed hydrogel at −4° C. to 37° C. for 15 minutes to 25 minutes.

13. A three-dimensional cell culture support having a double crosslink, including a structure gelated in a three-dimensional shape and manufactured according to the method of claim 1.

14. A casting tray for manufacturing a three-dimensional cell culture support, comprising:
a tray part formed in a plate shape and including a groove extending in a longitudinal direction of the plate shape in the inside of the plate shape, wherein a gel solution is accommodated in the groove; and
a mold part formed in a plate shape and covering the tray part after the gel solution is accommodated in the groove of the tray part,
wherein the mold part comprises a mold protrusion protruding toward the groove from the mold part and inserted into the groove when the mold part covers the tray part.

15. The casting tray of claim 14, wherein the mold protrusion is formed by combining at least one shape among a polyhedron, a cone, a cylinder, a hemisphere, and a sphere shape.

16. The casting tray of claim 15, wherein the mold protrusion comprises an embossed part embossed in a surface of the mold protrusion or an engraved part engraved in the surface of the mold protrusion.

17. The casting tray of claim 16, wherein the embossed part and the engraved part are formed in a spiral shape.

18. Time casting tray of claim 14, wherein, in time groove of the tray part, a tray protrusion protruding from the groove is formed.

19. The casting tray of claim 14, wherein, in time groove of the tray part, a tray groove engraved in a predetermined shape from the groove is formed.

20. The casting tray of claim 14, wherein the mold part is provided as a plurality of mold parts having different shapes of mold protrusions, and
the plurality of mold parts cover the tray part sequentially.
